(19) United States
(12) Patent Application Publication
(10) Pub. No.: US 2004/0112366 A1
(43) Pub. Date: Jun. 17, 2004

(54) Thermal device

(75) Inventors: Michael C. Addison, Newcastle upon Tyne (GB); Graeme D. Cruickshank, Newcastle upon Tyne (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/662,551

(22) Filed: Sep. 15, 2003

Related U.S. Application Data
(63) Continuation of application No. PCT/US02/07917, filed on Mar. 15, 2002.

(30) Foreign Application Priority Data
Mar. 16, 2001 (GB) 0106519.2

(51) Int. Cl. A61F 7/00

(52) U.S. Cl. 126/204; 126/206; 126/263.03

(57) ABSTRACT

A portable, self-heating thermal cushion for use in conjunction with a seating surface to provide a heated and thermally insulated seating region of sufficient size to extend underneath the buttocks of the user, the cushion being in the form of a composite laminated structure comprising a backsheet, a topsheet and an intermediate layer of a heat generating material, said intermediate layer being in thermal contact with the seating region of the cushion so as to provide a heating region generally coextensive with the seating region for warming the buttocks of the user.

### CROSS REFERENCE TO PRIOR APPLICATION

This is a continuation of International Application PCT/US02/07917, with an international filing date of Mar. 15, 2002, and published in English.

### TECHNICAL FIELD

The present invention relates to thermal devices. In particular, the invention relates to a portable, self-heating thermal cushion for use in conjunction with a seating surface for providing safe, even and effective warming of the buttocks of the user.

### BACKGROUND OF THE INVENTION

Thermal packs for body warmers are well known in the art, particularly those based on a self-heating mixture of a reactive iron powder and activated carbon. Such packs require oxygen to produce a controlled exothermic reaction, capable of generating heat over a period of several hours. Typically, the mixture is packaged in an air permeable pocket, which is sealed in an impermeable outer pouch ready for use.

The use of self-heating thermal packs for providing heated seating is known in the art. In particular U.S. Pat. No. 4,604,987 discloses a thermal cushion comprising a heating pack of the type disclosed in U.S. Pat. No. 1,613,120 or U.S. Pat. No. 1,953,513. Thermal cushions as described in the art are prone to a number of problems, however, including inadvertent leakage of the heat generating material and difficulty in controlling the rate of heat generation. Furthermore such cushions have been found to be deficient owing to the very limited region of the cushion that will actually be in thermal contact with the heating medium.

It is an object of the present invention to provide a portable self-heating thermal cushion providing safe, even and effective heating of the buttocks of the user.

Another object of the invention is to provide a disposable thermal cushion formed from an inexpensive composite laminate material.

A further object of the invention is to provide a cushion design providing rapid heat generation but which at the same time ensures safe and even heating across the entire region of the buttocks in contact with the cushion.

A further object of the invention is to provide a cushion design providing rapid heat generation but which at the same time ensures the lay-out of the heating cells within the composite structure are adequate to ensure consistent and even heating across the entire buttock region contacting the cushion.

Yet another object of the invention is to ensure the cushion continues to function when wet and is of sufficiently robust construction to prevent leakage of the thermal materials.

### SUMMARY OF THE INVENTION

According to a first aspect of the invention, there is provided a portable, self-heating thermal cushion for use in conjunction with a seating surface to provide a heated and thermally insulated seating region of sufficient size to extend underneath the buttocks of the user. The cushion is generally in the form of a composite laminated structure comprising a backsheet, a topsheet and an intermediate layer of a heat generating material. The cushion is designed such that the intermediate layer is in thermal contact with the seating region of the cushion so as to provide a heating region generally coextensive with the seating region for warming the buttocks of the user.

The seating region of the cushion is understood herein to be the region of the cushion that in normal use thereof contacts the user's buttocks. The size of the seating region (sometimes referred to herein as ‘the seating area’) can be defined in terms of the area of contact with the user's buttocks. While the actual area of contact can vary from individual to individual, preferably the seating region provides a minimum area of contact (or minimum seating area) for the target user population (male, female, lightweight, medium, heavyweight, etc) of at least about 200 cm², preferably at least about 400 cm², more preferably at least about 500 cm². In preferred embodiments herein, the shape and size of the cushion is such that essentially its entire upper surface contacts the user's buttocks, so that the size of the seating region is effectively the same as the area of the upper surface of the cushion.

The cushion is designed such that the intermediate layer of heat generating material is in direct or indirect thermal contact with the seating region, this thermal contact and resulting elevated temperature defining the so-called ‘heating region’ of the cushion. Preferably, thermal contact is over at least about 30% and preferably over at least about 50% of the cushion's seating region, in which case the heating region is said to be generally coextensive with the seating region. In terms of size, the area of the seating region in thermal contact with the intermediate layer and/or at elevated temperature (this area sometimes referred to herein as ‘the heating area’) is preferably at least about 150 cm², more preferably at least about 250 cm², and especially at least about 340 cm².

The thermal characteristics of the intermediate layer and the heat transfer characteristics from the intermediate layer to the seating region are preferably such as to ensure that in either actual or simulated use, the temperature of at least about 50% and preferably at least about 70% of the heating region is raised by at least about 10° C. above ambient temperature for a period of at least about 45 minutes, preferably at least about 90 minutes and especially at least about 120 minutes. For screening purposes, the in-use thermal characteristics of the cushion can be simulated using the temperature profile test described hereinbelow. Following the simulated-use test procedure, the temperature of at least about 50% and preferably at least about 70% of the heating region of the cushions of the invention is raised initially by at least about 10° C., preferably at least about 15° C. and more preferably at least about 18° C. above ambient temperature (20° C.) and subsequently reaches a plateau temperature of at least about 10° C. and preferably at least about 12° C. above ambient temperature.

The cushions of the invention are designed to reach their operating temperature rapidly as well as to deliver a consistent and even generation of warmth. In preferred embodiments, the temperature increase of 10° C. or more, preferably 15° C. or more, is achieved within about 15 minutes and preferably within about 6 minutes of the commencement of heat generation. Moreover, the plateau temperature preferably lasts for a period of at least about 45 minutes, more preferably at least about 90 minutes and especially at least about 120 minutes from commencement of heat generation.

The cushions can be constructed in any suitable manner, but preferably the backsheet and topsheet are secured to one another around the periphery of the seating region and also by one or more seals within the seating region so as to maintain the physical stability of the intermediate layer. The cushion, moreover, is preferably formed with a plurality of spaced reservoirs for holding the heat generating material in so-called ‘heating cells’, the area of each reservoir (projected and viewed from above) generally being from about 1 cm² to about 50 cm and preferably from about 5 cm² to about 30 cm² with a reservoir to non-reservoir area ratio of from about 100:1 to about 1:100, preferably from about 100:1 to about 1:2. The depth of the reservoirs is preferably less than about 2 cm, more preferably less than about 1 cm and especially from about 0.1 to 0.3 cm and each reservoir has a capacity of from about 0.1 to about 100 grams and preferably from about between 0.5 and 10 grams of heat generating material. Reservoir dimensions are measured with the cushion in its normal relaxed condition. The number of reservoirs on the other hand is preferably at least 2 or more, more preferably from about 10 to about 400, yet more preferably from about 15 to about 150, and especially from about 20 to about 80, and more especially from about 25 to about 40.

The simulated-use temperature profile test is undertaken at an ambient temperature of 20° C. in the following manner. The cushion is placed on a laboratory bench with three or more temperature probes taped in the central region of the seating surface in close proximity to the heat generating material (each probe, for example, being directly above and in contact with a heating cell) so as to provide temperature readings over an area corresponding to at least about 50% and preferably at least about 70% of the heating region. A lidded plastic bucket containing 10 litres of water at 35° C. is then placed on top of the cushion and temperature probes to simulate a person sitting on the cushion. The plastic bucket has a bottom diameter of 21 cm, a top diameter of 29 cm, a height of 28 cm and a contact area of 346 cm². Temperature data are then collected at minute intervals over a period of two hours and the average temperature readings plotted against time. Over the course of the test, the temperature of the water in the bucket drops to about 33° C. after 60 minutes and to about 31° C. after 120 minutes.

Heat generating materials suitable for use herein are of various types and include materials capable of generating heat by the initiation of a phase change within the material and materials which comprise two of more reactive components which are capable of generating heat during mixing thereof, for example, hydratable metal oxide salts which generate heat on reaction with water. In preferred embodiments, however, the heat generating material is a material capable of generating heat on exposure to air.

Highly suitable heat generating materials of this type is a composition comprising from about 30% to about 80% by weight of iron powder, from about 3% to about 25% by weight of carbonaceous material comprising activated carbon, non activated carbon or a mixture thereof, from about 0.5% to about 10% by weight of a metal salt, from about 1% to about 40% by weight of water and from about 0.1% to about 10% by weight of a reducing agent such as sodium thiosulphate. The composition is activated by exposure to air and to this end the topsheet of the cushion preferably comprises or is fabricated from an air-permeable material effective in controlling the flow of air to the heat generating material. In preferred embodiments, the topsheet and backsheet materials and construction are such that air flow is controlled solely or largely by the permeability of the topsheet.

The cushions of the invention are preferably constructed with a waterproof or water-resistant backsheet and/or topsheet in order to enable functioning under wet, damp or moist conditions. The backsheet itself is preferably made of a cushioned material such as neoprene, a material that also provides valuable thermal insulation. Alternatively, the cushion can include a separate layer of cushioning on the side of the intermediate layer opposite the seating region.

The topsheet, on the other hand, preferably comprises a microporous laminated nonwoven film, highly suitable nonwoven films having a moisture vapour transmission rate of from about 100 to about 5000 g/m²/24 hours and preferably from about 1000 to about 2000 g/m²/24 hours. Moisture vapour transmission rate is suitably measured as follows. An upright cup is filled to 6 mm below the rim of the cup with calcium chloride dessicate of a size greater than a No 10 seive. A sample of film is then secured across the top of the cup using a retaining ring and gasket and the unit is placed in a controlled oven at 40° C. and 75% relative humidity for 5 hours. The weight gain is measured and expressed as the average of three replicates rounded to the nearest 100 g/m²/24 hours.

In a method aspect, the present invention provides a method of warming the buttocks of a user while seated on a seating surface such as a seat, bench, chair or ground, the method comprising placing a portable, self-heating thermal cushion as described herein on the seating surface prior to the user sitting thereon. In use of the cushions, the seating region is preferably raised to a temperature in the range from about 25° C. to about 50° C. and more preferably from about 32° C. to about 39° C. and maintained at a temperature above ambient for a period of at least about 45 minutes and preferably at least about 90 minutes.

### BRIEF DESCRIPTION OF THE DRAWINGS

The invention will now be described by way of example with reference to the accompanying drawings in which:

FIG. 1 is a plan view of a thermal cushion according to the invention;

FIG. 2 is a cross-sectional view of the cushion of FIG. 1 taken along X—X; and

FIG. 3 is an expanded view of a portion of the cross-section of FIG. 2.

### DETAILED DESCRIPTION OF THE INVENTION

The self-heating thermal cushion of the invention is in the form of a composite laminated structure comprising a backsheet and a topsheet, these usually but not necessarily being formed of materials of different composition and/or construction. Sandwiched between the backsheet and topsheet is a layer of heat generating material arranged in one or more ‘heat cells’ whereby the construction and thermophysical properties of the heat-generating layer and topsheet are such as to provide significant warming of the buttock region of a user seated on the cushion. If desired, additional intermediate layers or portions of a non-heat generating but preferably heat-conducting material is sandwiched between the backsheet and topsheet. As used herein, the term backsheet is understood to be the layer normally contacting the seat during use. The topsheet, on the other hand, is understood to be the layer normally contacting the user during use.

Preferably, the cushion comprises a plurality of heat cells arranged in such a way as to provide adequate heating over the entire buttocks region of the user. Leakage of heat-generating material between heating cells or from the cushion is prevented by securing the top and bottom sheets together around the periphery of the seating region and also by one or more seals within the seating region so as to physically isolate the heat cells from on another. To minimise unnecessary wastage of materials, it is recommended the sealing area between cells is restricted in size to no greater than about twice that of the heating area of the device and preferably no greater than the heating area of the device. Further it is preferred that the seating area of the cushion is greater than about 200 sq.cm and is preferably greater than about 400 sq.cm. In order to optimise contact and heat transfer properties, it is recommended the geometry of the top layer is flat.

The portable thermal cushion of the present invention provides consistent, convenient and comfortable heat to the buttocks region of the user while retaining sufficient structural integrity to prevent leakage from the heat cells to the environment. ‘Portable’ as used herein, means that the size and weight of the cushion structure are such that the user can easily carry the cushion on their person to the point of use, for example, in the form of a wallet-sized package. Further the heating action can be initiated when and where desired by the user without recourse to an external power source.

‘Adequate heating’ as used herein means that in actual or simulated use, the heat-generating layer has sufficient power as to raise the seating region in thermal contact therewith from an ambient temperature of 20° C. to at least about 30° C. on activation, preferably to at least about 35° C. and more preferably to at least about 38° C. Preferably, this increase in temperature is achieved over at least about 50% and preferably at least about 70% of the heating region, over at least about 30%, preferably over at least about 50% of the seating region and within about 15 minutes of activation of the device, ideally within about 6 minutes.

The reservoir or reservoirs are preferably formed as indentations in the backsheet or topsheet or in an intermediate non-heat generating layer, where present, by positive pressure applied by a punch or by a reduction in pressure on the underside of the layer to be indented effected via a suctioning device. Quantities of the heat generating material are then introduced into the reservoir indentations and thereafter non-deformed portions of the topsheet and backsheet are secured to one another such that the resulting reservoirs remain sealed and completely isolated from all other reservoirs during storage, transport and use.

The heat generating material for use herein can comprise any suitable material or composition capable of generating heat, such as compositions that generate heat by exothermic reaction, heat of crystallisation and other phase transfer compositions, etc. Preferably, the heat generating material takes the form of a loose packed, particulate exothermic composition contained in an amount such as to partially fill the available reservoir volume of each heating cell.

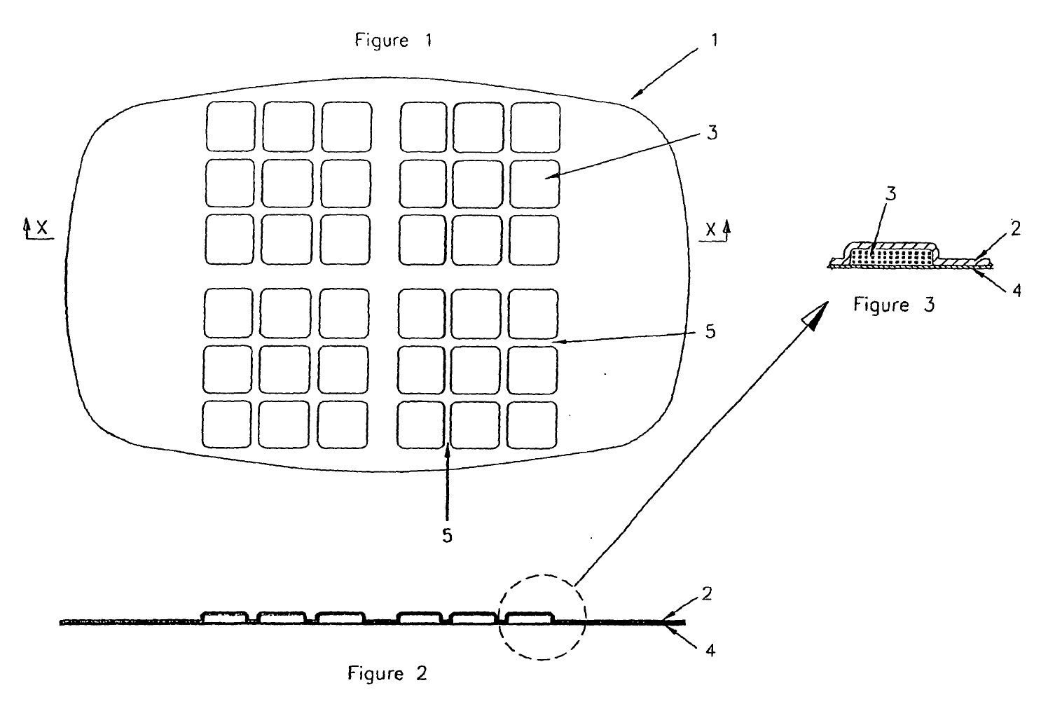

Referring to FIG. 1, portable heated cushion (1) comprises backsheet (2) formed of a flexible, deformable material that will normally be in contact with the seat during use. Backsheet (2) has formed within it a plurality of reservoirs (3)—in the illustrated embodiment 36 in total, each 3 cm square arranged in four groups over an area of 21.6 cm square—into which quantities of the heat generating mixture have been introduced. Topsheet (4) is then affixed to backsheet (2) around the periphery of the cushion and along seal lines (5) so as to isolate the reservoirs from one another. The size of the resulting cushion (and seating region) is approximately 820 cm². Topsheet (4) and backsheet (2) may be made of any suitable flexible, deformable material including but not limited to wovens, knits, films, foams and nonwovens including spunbond, carded, meltblown, hydroentangled, through-air bonded, air laid and wet laid. These materials may be made from natural fibres including but not limited to cotton, wool, linen or manmade polymeric materials such as polypropylene, polyester, nylon, polyethylene, metallocene catalyst polyethylene, and the like. A material that has been found to be particularly suitable for backsheet (2) is ethylene vinylacetate as supplied by Eurofoam. A material that has been found to be particularly suitable for topsheet (4) is a microporous laminated nonwoven polypropylene film as supplied by Tredegar (MVTR 1500).

The heat generating material can comprise any composition capable of generating heat by exothermic reaction or phase-transfer. However, the heat generating material preferably comprises a mix of chemical compounds that undergo an oxidation reaction during use, the mix preferably being in particulate form. Alternatively the heat generating material can be formed into agglomerated granules or directly compacted into compaction forms such as granules, pellets, tablets and/or slugs and mixtures thereof. The mix of compounds typically comprises iron powder, carbon, a metal salt(s) and water. Mixtures of this type react when exposed to oxygen to provide heat for several hours.

Suitable sources of iron powder include cast iron powder, reduced iron powder, electrolytic iron powder, scrap iron powder, pig iron, wrought iron, various steels, iron alloys and the like and treated varieties of these iron powders. There is no particular limitation as to purity, kind, etc. as long as the iron powder can be used to generate heat in the presence of electrically conducting water and air. Typically, the iron powder comprises from about 30% to about 80% by weight, preferably from about 50% to about 70% by weight, of the heat generating composition.

Active carbon sources suitable for use herein include coconut shell, wood, charcoal, coal, bone coal, etc and also animal products, natural gas, fats, oils and resins. There is no limitation to the kinds of active carbon used, however, the preferred active carbon has superior water holding capabilities and the different carbons may be blended to reduce cost. Non-activated carbon sources can also be used herein. Typically, activated carbon, non activated carbon, and mixtures thereof, comprises from about 3% to about 25%, preferably from about 8% to about 20%, most preferably from about 9% to about 15% by weight, of the heat generating composition.

The metal salts useful herein include sulfates such as ferric sulfate, potassium sulfate, sodium sulfate, manganese sulfate, magnesium sulfate, and chlorides such as cupric chloride, potassium chloride, sodium chloride, calcium chloride, manganese chloride, magnesium chloride and cuprous chloride, and mixtures thereof. Also, carbonates, acetates, nitrates, nitrites and other salts can be used. The preferred metal salts are sodium chloride, cupric chloride, and mixtures thereof. Typically, the metal salt(s) comprises from about 0.5% to about 10% by weight, preferably from about 1.0% to about 5% by weight, of the heat generating exothermic composition.

The water used herein can be from any appropriate source. There is no particular limitation to purity, kind etc. Typically, water comprises from about 1% to about 40% by weight, preferably from about 10% to about 30% by weight, of the heat generating composition.

Additional water holding materials can also be added as appropriate. Suitable additional water holding materials include vermiculite, porous silicates, wood powder, wood flour, cotton cloth having a large amount of fluffs, short fibers of cotton, paper scrap, vegetable matter, super absorbent water swellable or water soluble polymers and resins, carboxymethylcellulose salts, and other porous materials having a large capillary function and hydrophilic property. Typically, the additional water holding materials comprise from about 0.1% to about 30% by weight, preferably from about 0.5% to about 20% by weight, most preferably from about 1% to about 10% by weight, of the heat generating composition.

Other additional components include agglomeration aids such as gelatin, natural gums, cellulose derivatives, cellulose ethers and their derivatives, starch, modified starches, polyvinyl alcohols, polyvinylpyrrolidone, sodium alginates, polyols, glycols, corn syrup, sucrose syrup, sorbitol syrup and other polysaccharides and their derivatives, polyacrylamides, polyvinyloxoazolidone, and maltitol syrup, dry binders such as maltodextrin, sprayed lactose, cocrystallixed sucrose and dextrin, modified dextrose, sorbitol, mannitol, microcrystalline cellulose, microfine cellulose, pre gelatinised starch, dicalcium phosphate, and calcium carbonate, oxidation reaction enhancers such as elemental chromium, manganese, or copper, compounds comprising said elements, or mixtures thereof, hydrogen gas inhibitors such as inorganic or organic alkali compounds or alkali weak acid salts including sodium hydroxide, potassium hydroxide, sodium hydrogen carbonate, sodium carbonate, calcium hydroxide, calcium carbonate, sodium propionate and sodium thiosulphate; fillers such as natural cellulosic fragments including wood dust, cotton linter, and cellulose, synthetic fibers in fragmentary form including polyester fibers, foamed synthetic resins such as foamed polystyrene and polyurethane, and inorganic compounds including silica powder, porous silica gel, sodium sulfate, barium sulfate, iron oxides, and alumina; and anti caking agents such as tricalcium phosphate and sodium silicoaluminate. Such components also include thickeners such as corn starch, potato starch, carboxymethylcellulose , and α-starch, and surfactants such as those included within the anionic, cationic, non-ionic, zwitterionic, and amphoteric types. The preferred surfactant, if used however, is non-ionic. Still other additional components include extending agents such as metasilicates, zirconium, and ceramics.

Preferred herein are heat generating materials in particulate form and preferably, at least 50%, more preferably 70%, even more preferably 80% and most preferably 90% by weight of the particles of the heat generating material have a mean particle size of less than 200 μm, preferably less than 150 μm.

The above-mentioned components of the composition are blended using conventional blending techniques. Suitable methods of blending these components are described in detail in U.S. Pat. No. 4,649,895 to Yasuki et al., issued Mar. 17, 1987.

Heat cells comprising the above described components are typically formed by adding a fixed amount of the heat generating material in particulate form to reservoirs formed in the backsheet layer. The topsheet layer is then placed over the backsheet layer and the two are then bonded together, preferably using a low heat to form a unified laminate structure. Preferably each heat cell reservoir has a similar volume of heat generating material and has similar oxygen permeability means. However the volume of the heat generating material, shape of the heat cell, and oxygen permeability may be different from heat cell to heat cell as long as the resulting cell temperatures generated are within accepted safety ranges for their intended use.

The heat cell reservoirs can have any geometric shape e.g. disk, triangle, pyramid, cone, sphere, square, cube, rectangle, rectangular parallelepiped, cylinder, ellipsoid, and the like. The preferred shape of the heat cell reservoirs is a square of dimensions from about 0.2 to about 10cm, preferably about 1 to about 7 cmand most preferably from about 2 to about 5 cm. The heat cell reservoirs have a depth as made of less than about 2 cm, preferably less than 1 cm and most preferably from about 0.1 to 0.3 cm. The ratio of fill volume to reservoir volume of the heat cell reservoirs is from about 0.1 to 1 preferably less than about 0.7, more preferably less than about 0.5.

Oxygen permeability can be achieved by selecting materials having the requisite permeability properties for the backsheet, topsheet or optional intermediate layers. The desired permeability properties may be provided by the use of a microporous film. A preferred microporous film, which offers excellent air flow management, is Tredegar 1500MVTR. This is a polypropylene film that is modified to allow ‘breathability’, a feature useful in applications such as diapers (see U.S. Pat. No. 5,876,393). This controlled permeability may be achieved by the addition of some inorganic or filler material to the polyolefin melt prior to film formation, then applying elongational stresses to the film, which imparts differential deformation through the film in the vicinity of the inert filler material, such that a microporous structure is created thus imparting the property of breathability to the film (see e.g. U.S. Pat. No. 5,733,628).

Alternatively, the desired permeability properties may be provided by the use of films that have pores or holes formed therein. The formation of these holes/pores can be via extrusion, cast/vacuum formation or by hot needle aperturing. The size of the apertures is preferably about 0.127 mm diameter, and there are preferably 25 to 40 apertures per heat cell reservoir. Another preferred method of making apertures is to pierce the cell-covering layer with cold needles. Alternatively, apertures can be produced by vacuum forming or using a high-pressure water jet forming process. Oxygen permeability can also be provided by perforating at least one of the backsheet and topsheet with aeration holes using, for example, an array of pins having tapered points and diameters of from about 0.2 mm to about 2 mm, preferably from about 0.4 mm to about 0.9 mm. The array of pins is patterned such that the backsheet and/or topsheet are perforated by from about 10 to 30 pins per square centimetre. Alternatively, after the backsheet and topsheet have been bonded together, enclosing the heat generating material in the reservoir between them, one side of the heat cell reservoir can be perforated with aeration holes using, for example, at least one pin, preferably an array of from about 20 to about 60 pins having tapered points and diameters of from about 0.2 mm to about 2 mm, preferably from about 0.4 mm to about 0.9 mm. The pins are pressed through one side of the backsheet and/or topsheet to an extent of from about 2% to about 100%, preferably from about 20% to about 100%, and more preferably from about 50% to about 100% of the depth of the heat generating composition

A further approach has been disclosed in WO-A-97/33542 which discloses a means for controlling oxygen flux whereby an oxygen-permeable substrate is coated with an oxygen-permeable silicone-based elastomer to a thickness which regulates the oxygen flux through the material.

The finished heated cushion is typically packaged in a secondary package. An air impermeable package can be used to prevent premature oxidation reaction as described in U.S. Pat. No. 4,649,895. Alternatively, other means can also be used to prevent premature oxidation reaction, such as air impermeable removable adhesive strips placed over the aeration holes in the heat cell reservoirs such that, when the strips are removed, air is allowed to enter the reservoirs, thereby activating the oxidation reaction in the iron powder.

Following the temperature profile test procedure described hereinabove, a cushion according to the invention reaches a temperature of about 39° C. within about 5-6 minutes and plateaus at a temperature of about 33° C. after about 12 minutes, which temperature is maintained for the whole test period of 120 minutes. When similarly tested, an identical cushion except that it has already been activated and used and no longer generates heat, is found to have an initial temperature of about 28° C., gradually cooling over the period of the test to a final temperature of about 26° C. The cushion of the invention thus provides a temperature increase of at least about 5° C. as compared with the equivalent non-heating cushion for a period of at least 120 minutes.

The present invention also comprises a method for providing warmth when and where the user is required to sit for substantial periods in a cold environment. The method comprises the user placing the invention on the seating surface prior to the user sitting thereon. The method comprises maintaining a skin temperature of the bottom (buttocks) region of the user in the range 25C to about 50C. and preferably from about 32C to 39C.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed:

1. A portable, self-heating thermal cushion having a thermally insulated seating region, said cushion comprising a composite laminated structure, said structure comprising a backsheet, a topsheet and an intermediate layer comprising a heat generating material, said intermediate layer being in thermal contact with said seating region of said cushion, wherein said cushion is contacted with a seating surface, and wherein said seating region is of sufficient size to extend underneath the buttocks of the user to provide a heating region generally coextensive with said seating region for warming said buttocks.

2. The cushion of claim 1, wherein said seating region has a contact area of at least about 200 cm².

3. The cushion of claim 1, wherein said intermediate layer has at least about 30% thermal contact with said seating region.

4. The cushion of claim 1, wherein said thermal layer has thermal characteristics and heat transfer characteristics, said thermal and heat transfer characteristics providing a heating region, said thermal and heat transfer characteristics being selected to raise the temperature of said heating region by at least 10° C. above ambient temperature for a period of at least about 45 minutes.

5. The cushion of claim 4, wherein said cushion has a simulated-use temperature profile such that the temperature of at least about 50% of said heating region is raised by at least about 10° C. above ambient temperature within about 15 minutes of the commencement of heat generation and wherein a plateau temperature of at least about 10° C. above ambient temperature is maintained for a period of at least about 45 minutes from the commencement of heat generation.

6. The cushion of claim 1, wherein said backsheet and said topsheet are secured to one another around a periphery of said seating region.

7. The cushion of claim 1, further comprising a plurality of spaced reservoirs for holding said heat generating material and wherein the area of each of said reservoirs ranges from about 1 cm² to about 50 cm².

8. The cushion of claim 7, wherein each of said reservoirs has a depth of less than about 2 cm.

9. The cushion of claim 8, wherein each of said reservoirs has a capacity ranging from about 0.1 grams to about 100 grams.

10. The cushion of claim 7, wherein said cushion has a reservoir to non-reservoir area ratio ranging from about 100:1 to about 1:100.

11. The cushion of claim 1, wherein said heat generating material is a material capable of generating heat on exposure to air.

12. The cushion of claim 11, wherein said heat generating material comprises from about 30% to about 80% by weight of iron powder, from about 3% to about 25% by weight of carbonaceous material comprising activated carbon, non activated carbon or a mixture thereof, from about 0.5% to about 10% by weight of a metal salt, from about 1% to about 40% by weight of water, and from about 0.1% to about 10% by weight of a reducing agent.

13. The cushion of claim 11, wherein said topsheet comprises an air-permeable material effective in controlling the flow of air to said heat generating material

14. The cushion of claim 1, wherein said heat generating material is a material capable of generating heat by the initiation of a phase change within said material.

15. The cushion of claim 1, wherein said heat generating material comprises two or more reactive components capable of generating heat during mixing thereof.

16. The cushion of claim 1, wherein said cushion further comprises a waterproof or water-resistant backsheet and/or topsheet.

17. The cushion of claim 1, wherein said cushion comprises a separate layer of cushioning on the side of said intermediate layer opposite said seating region.

18. The cushion of claim 1, wherein said backsheet comprises a thermally-insulating material.

19. The cushion of claim 1, wherein said topsheet comprises a microporous laminated nonwoven film and wherein said film has a moisture vapour transmission rate ranging from about 100 g/m²/24 hours to about 5000 g/m²/24 hours.
